(19) United States
(12) Patent Application Publication
(10) Pub. No.: US 2012/0197405 A1
(43) Pub. Date: Aug. 2, 2012

(54) INTERVERTEBRAL IMPLANT

(75) Inventors: Walter Cuevas, Irvine, CA (US); Robert Flower, Sun City, CA (US); Fausto Olmos, Laguna Niguel, CA (US); Christopher Warren, Aliso Viejo, CA (US); Peter Davis, Dana Point, CA (US)

(73) Assignee: INTERVENTIONAL SPINE, INC., Irvine, CA (US)

(21) Appl. No.: 13/358,280

(22) Filed: Jan. 25, 2012

Related U.S. Application Data
(60) Provisional application No. 61/438,046, filed on Jan. 31, 2011.

(51) Int. Cl. A61F 2/44

(52) U.S. Cl. 623/17.16

(57) ABSTRACT

An intervertebral implant and related methods of use are provided for treatment of spaces between two vertebrae. The implant can comprise a first member and a second member that are configured for engagement in a stacked configuration. The first member and second member can be inserted separately so that the intervertebral space can be reached through limited access pathways. The first member and second member can be coupled in situ in the intervertebral space to form an implant of desired height. In this manner, an intervertebral implant having final dimensions that would not fit through a limited access pathway can be implanted through the access pathway by inserting the implant in separate member pieces.

### PRIORITY

The present application claims the benefit of U.S. Provisional Application No. 61/438,046, filed Jan. 31, 2011, the disclosure of which is hereby incorporated by reference herein in its entirety.

### BACKGROUND

1. Field of the Disclosure

The present application relate to medical devices and, more particularly, to a medical device for the spine.

2. Description of the Related Art

The human spine is a flexible weight bearing column formed from a plurality of bones called vertebrae. There are thirty-three vertebrae, which can be grouped into one of five regions (cervical, thoracic, lumbar, sacral, and coccygeal). Moving down the spine, there are generally seven cervical vertebrae, twelve thoracic vertebrae, five lumbar vertebrae, five sacral vertebrae, and four coccygeal vertebrae. The vertebrae of the cervical, thoracic, and lumbar regions of the spine are typically separate throughout the life of an individual. In contrast, the vertebra of the sacral and coccygeal regions in an adult are fused to form two bones, the five sacral vertebrae which form the sacrum and the four coccygeal vertebrae which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. Thus, spinal fusion is the process by which the damaged disc is replaced and the spacing between the vertebrae is restored, thereby eliminating the instability and removing the pressure on neurological elements that cause pain.

Spinal fusion can be accomplished by providing an intervertebral implant between adjacent vertebrae to recreate the natural intervertebral spacing between adjacent vertebrae. Once the implant is inserted into the intervertebral space, osteogenic substances, such as autogenous bone graft or bone allograft, can be strategically implanted adjacent the implant to prompt bone ingrowth in the intervertebral space. The bone ingrowth promotes long-term fixation of the adjacent vertebrae. Various posterior fixation devices (e.g., fixation rods, screws etc.) can also be utilize to provide additional stabilization during the fusion process.

Notwithstanding the variety of efforts in the prior art described above, these intervertebral implants and techniques are associated with another disadvantage. In particular, these techniques typically involve an open surgical procedure, which results in higher cost, lengthy in-patient hospital stays and the pain associated with open procedures. In addition, many intervertebral implants are inserted anteriorly while posterior fixation devices are inserted posteriorly. This results in additional movement of the patient.

Therefore, there remains a need in the art for an improved intervertebral implant. Preferably, the implant is implantable through a minimally invasive procedure. Further, such devices are preferably easy to implant and deploy in such a narrow space and opening while providing adjustability and responsiveness to the clinician.

### SUMMARY

While using minimally invasive procedures to deploy an intervertebral prostheses is generally advantageous, such procedures do have the disadvantages of generally requiring the device to be passed through a relatively small diameter passage or tube. In addition, deployment tools typically must also be deployed through the small diameter passage or tube.

As described, an intervertebral implant is typically limited in size by the size of the passage or tube through which the implant must fit to reach the intervertebral space. Some intervertebral implants have tried to solve this problem by creating an expandable implant. However, these implants required complicated and/or large deployment tools. In this regard, according to at least one of the embodiments disclosed herein is the realization that an intervertebral implant is needed that can fit through small passages and be deployed simply and easily to fit in an intervertebral space.

Therefore, in accordance with at least one of the embodiments disclosed herein, there is provided an implant for use of intervertebral endoscope that overcomes the aforementioned drawbacks. For example, the intervertebral implant can have a collapsed configuration that can fit through small openings and then expanded in a deployed configuration to fit in an intervertebral space. Further, the implant can be collapsed after installation, which allows the implant to be extracted or adjusted in the event of incorrect placement. In some embodiments, the intervertebral implant can at least partially be made of an allograft, such as cortical bone. In certain embodiments, the intervertebral implant can be made substantially or entirely of an allograft, such as cortical bone. In some embodiments, the body can be at least partially made of a biocompatible material, such as Polyether-etherketone (PEEK™) and can be an interbody cage.

More specifically, some embodiments disclosed herein comprise a method of implanting a stackable intervertebral implant. The method comprises inserting a first member made substantially of bone allograft of the implant into the disc cavity and inserting a second member made substantially of bone allograft of the implant into the disc cavity so that it slideably engages with the first member of the implant in a stacked configuration.

Some embodiments disclosed herein comprise an intervertebral implant that includes a first member comprising at least one channel extending along a longitudinal axis of the first member, the at least one channel being open to a top side and rear side of the first member, the first member further comprising a rear side having an angled surface. The implant can also include a second member comprising a bottom side having at least one rail extending along a longitudinal axis of the second member, the at least one rail configured for slideable engagement with the at least one channel, the second member further comprising a front side with an angled surface. The first and second members are formed substantially entirely of bone allograft.

Some embodiments disclosed herein comprise an intervertebral implant that includes a first member comprising at least one channel extending along a longitudinal axis of the first member and a second member comprising at least one rail extending along a longitudinal axis of the second member, the at least one rail configured for slideable engagement with the at least one channel.

### BRIEF DESCRIPTION OF THE DRAWINGS

The features of the devices and methods disclosed herein are described below with reference to the drawings. The illustrated embodiments are intended to illustrate, but not to limit the present application. The drawings contain the following figures:

FIG. 1 is a lateral elevational view of a portion of a vertebral column.

FIG. 2 is a posterior elevational view of the vertebral column of FIG. 1.

FIG. 3A is a superior plan view of a thoracic vertebra.

FIG. 3B is a lateral elevational view of a thoracic vertebra.

FIG. 4 is a superior plan view of a cervical vertebra.

FIG. 5 is a superior plan view of a lumbar vertebra.

FIG. 6A is a perspective top view of an intervertebral implant, according to an embodiment of the present application.

FIG. 6B is a side elevational view of the intervertebral implant of FIG. 6A.

FIG. 6C is a top plan view of the intervertebral implant of FIG. 6A.

FIG. 7 is a front cross-sectional elevational view taken at 7-7 in FIG. 6B.

FIG. 8 is a side cross-sectional elevational view taken at 8-8 in FIG. 6C.

FIG. 9A is a perspective top view of a lower member of the intervertebral implant of FIG. 6A.

FIG. 9B is a side elevational view of the lower member of FIG. 9A.

FIG. 9C is a top plan view of the lower member of FIG. 9A.

FIG. 10A is a perspective bottom view of an upper member of the intervertebral implant of FIG. 6A.

FIG. 10B is a side elevational view of the upper member of FIG. 10A.

FIG. 10C is a bottom plan view of the lower member of FIG. 10A.

FIG. 11 is a perspective top view of an intervertebral implant, according to an embodiment of the present application, in a collapsed configuration.

FIG. 12 is a perspective top view of a deployment tool, according to an embodiment of the present application, positioned adjacent an intervertebral space.

FIG. 13 is a side elevational view of the deployment tool of FIG. 12 and the intervertebral implant of FIG. 8 in an intervertebral space.

FIG. 14 is a side elevational view of the intervertebral implant of FIG. 11 in a first partially deployed configuration in an intervertebral space.

FIG. 15 is a side elevational view of the intervertebral implant of FIG. 11 in a second partially deployed configuration in an intervertebral space.

FIG. 16 is a side elevational view of the intervertebral implant of FIG. 8 in a deployed configuration in an intervertebral space.

### DETAILED DESCRIPTION OF THE PREFERRED EMBODIMENTS

In accordance with some embodiments disclosed herein, an intervertebral implant is provided that allows the clinician to insert the intervertebral implant through a minimally invasive procedure. For example, in one embodiment, one or more intervertebral implants can be inserted percutaneously to reduce trauma to the patient and thereby enhance recovery and improve overall results of the surgery. By minimally invasive, Applicant means a procedure performed percutaneously through an access device in contrast to a typically more invasive open surgical procedure. Such access devices typically provide an elongated passage that extends percutaneously through the patient to the target site. Examples of such access devices include, but are not limited to, endoscopes and the devices described in U.S. Patent Application Publication Nos. 2006-0030872 and 2005-0256525 and U.S. Pat. Nos. 6,793,656, 7,223,278 and co-pending U.S. Patent Application No. 13/245,130 filed Sep. 26, 2011 (Attorney Ref: TRIAGE.127A), the entireties of these patent applications and patents are hereby incorporated by reference herein.

In some embodiments, the intervertebral implant can ensure a minimum distance between adjacent vertebrae (a function that a healthy individual's intervertebral disc can performs naturally). Because embodiments of the intervertebral implant can be implemented through minimally invasive procedures, such embodiments of the implant can pass through the interior of an access device (usually a tube having a diameter of between 5-12 mm), and then expanded inside the patient. Further, the tools for deploying the implant should also be suitable for minimally invasive procedures.

Certain embodiments disclosed herein are discussed in the context of an intervertebral implant and spinal fusion because of the applicability and usefulness in such a field. The device can be used for fusion, for example, by expanding or configuring in situ the device to an appropriate intervertebral height and then inserting bone morphogenetic protein (BMP) or graft material. As such, various embodiments can be used to properly space adjacent vertebrae in situations where a disc has ruptured or otherwise been damaged. “Adjacent” vertebrae can include those vertebrae originally separated only by a disc or those that are separated by intermediate vertebra and discs. Such embodiments can therefore tend to recreate proper disc height and spinal curvature as required in order to restore normal anatomical locations and distances. However, it is contemplated that the teachings and embodiments disclosed herein can be beneficially implemented in a variety of other operational settings, for spinal surgery and otherwise.

In addition, certain embodiments of the device can also be used to provide dynamic intervertebral support. For example, the device can be used to maintain an intervertebral height without fusion and without disc degeneration to the adjacent levels. As discussed further herein, certain components of the device can be configured to resiliently support adjacent vertebrae. In some embodiments, the device can comprise one or more components fabricated from a resilient or elastic material. The device can thus be configured to deflect within a desired range of intervertebral heights in order to provide dynamic spacing and support between adjacent vertebrae.

It is contemplated that the implant can be used as an interbody or intervertebral device. The implant can be used in an intervertebral space or bone in order to fill the space or bone. In some embodiments, a biocompatible material, such as allograft, can be used in conjunction with the implant to fill the space.

Finally, the implant can also be introduced into the disc space anteriorly in an anterior lumbar interbody fusion (ALIF) procedure, posterior in an posterior lumbar interbody fusion (PLIF) or postero lateral interbody fusion, from extreme lateral position in an extreme lateral interbody fusion (XLIF) procedure, and transforaminal lumbar interbody fusion (TLIF), to name a few. Although the implant can be introduced from any of the directions described, it is especially advantageous for gaining access between the spinous processes in the posterior lumbar interbody fusion (PLIF) and transforaminal lumbar interbody fusion (TLIF) methods. In the case of transforaminal lumbar interbody fusion, it is contemplated that two implants can be used; one for each of the left and right transforaminal directions. See also co-pending U.S. patent application Ser. No. 13/245,130 filed Sep. 26, 2011 (Attorney Ref: TRIAGE.127A), which was incorporated by reference above for additional methods and apparatus for introducing the implant described herein.

It is contemplated that a number of advantages can be realized utilizing various embodiments disclosed herein. For example, as will be apparent from the disclosure, access to the intervertebral space can be realized through the posterior direction without cutting or distraction of the spine. Further, embodiments of the implant can enable sufficient restoration of the intervertebral space in order to properly restore disc function. Thus, normal anatomical locations, positions, and distances can be restored and preserved utilizing many of the embodiments disclosed herein.

Referring now to the figures, illustrations are provided for the purpose of illustrating some embodiments of the present application. However, the illustrated embodiments are intended to illustrate, but not to limit the present disclosure.

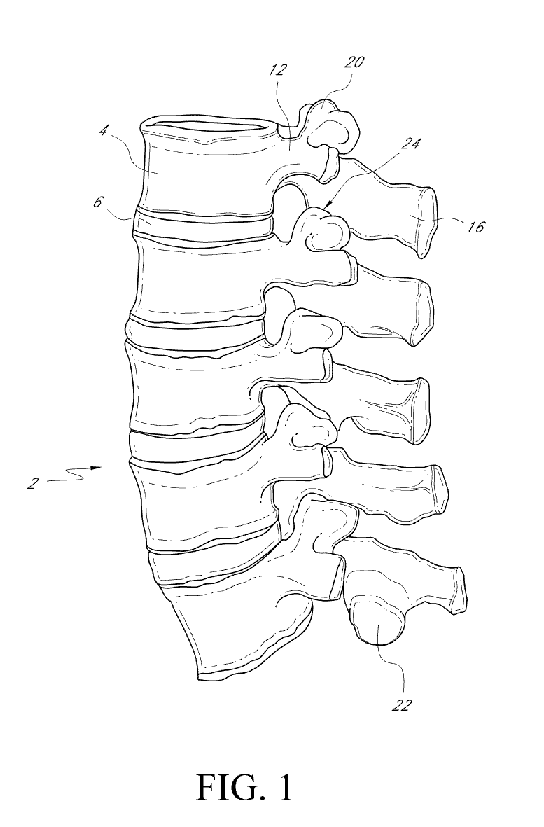

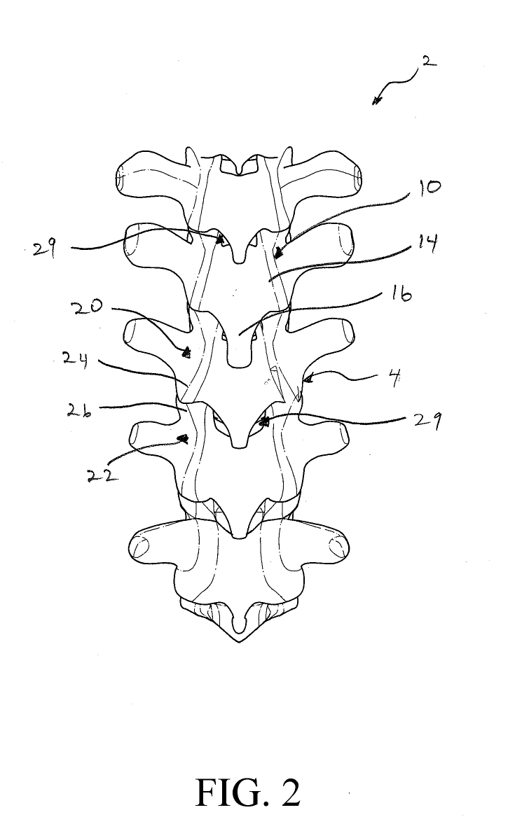

FIG. 1 is a lateral view of a vertebral column 2 and FIG. 2 is a posterior view of the vertebral column 2. As shown in FIGS. 1 and 2, the vertebral column 2 comprises a series of alternating vertebrae 4 and fibrous discs 6 that provide axial support and movement to the upper portions of the body. The vertebral column 2 typically comprises thirty-three vertebrae 4, with seven cervical (C1-C7), twelve thoracic (T1-T12), five lumbar (L1-15), five fused sacral (S1-S5) and four fused coccygeal vertebrae.

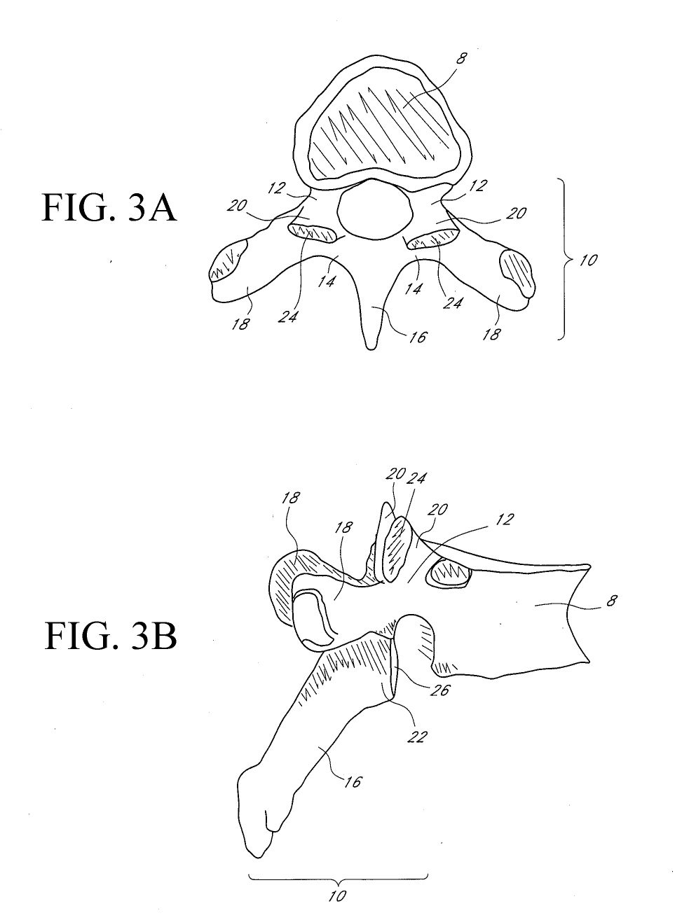

FIGS. 3A and 3B depict a typical thoracic vertebra. Each vertebra includes an anterior body 8 with a posterior arch 10. The posterior arch 10 comprises two pedicles 12 and two laminae 14 that join posteriorly to form a spinous process 16. Projecting from each side of the posterior arch 10 is a transverse 18, superior 20 and inferior articular process 22. The facets 24, 26 of the superior 20 and inferior articular processes 22 form facet joints 28 with the articular processes of the adjacent vertebrae.

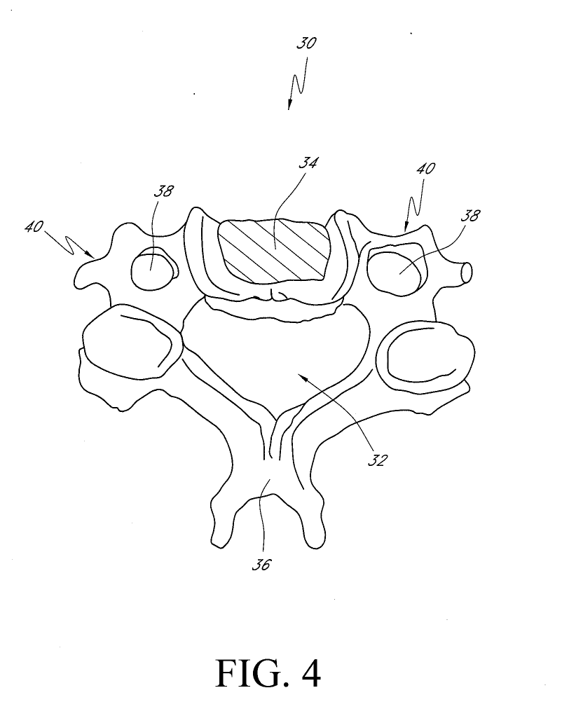

The typical cervical vertebrae 30, shown in FIG. 4, differ from the other vertebrae with relatively larger spinal canals 32, oval shaped vertebral bodies 34, bifid spinous processes 36 and foramina 38 in their transverse processes 40. These foramina transversaria 38 contain the vertebral artery and vein. The first and second cervical vertebrae also further differentiated from the other vertebrae. The first cervical vertebra lacks a vertebral body and instead contains an anterior tubercle. Its superior articular facets articulate with the occipital condyles of the skull and are oriented in a roughly parasagittal plane. The cranium is able to slide forward and backwards on this vertebra. The second cervical vertebra contains an odontoid process, or dens, which projects superiorly from its body. It articulates with the anterior tubercle of the atlas, forming a pivot joint. Side to side movements of the head occur at this joint. The seventh cervical vertebra is sometimes considered atypical since it lacks a bifid spinous process.

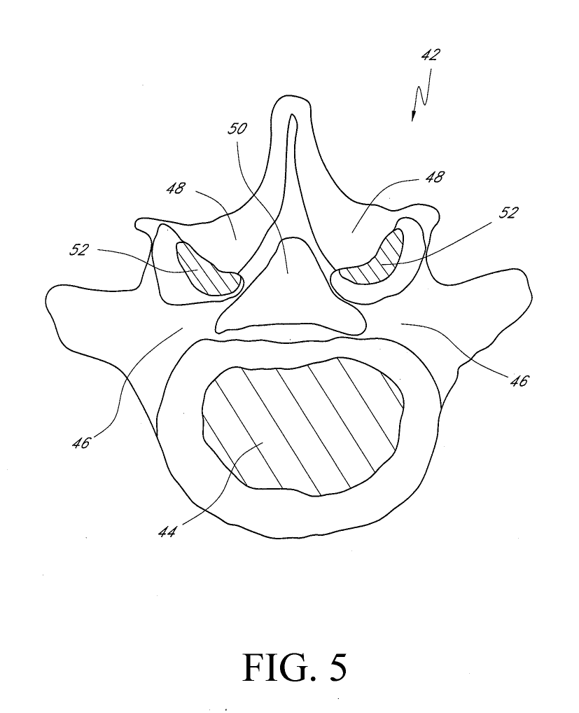

Referring to FIG. 5, the typical lumbar vertebrae 42 is distinguishable from the other vertebrae by the absence of foramina transversaria and the absence of facets on the surface of the vertebral body 44. The lumbar vertebral bodies 44 are larger than the thoracic vertebral bodies and have thicker pedicles 46 and laminae 48 projecting posteriorly. The vertebral foramen 50 is triangular in shape and larger than the foramina in the thoracic spine but smaller than the foramina in the cervical spine. The superior 52 and inferior articular processes (not shown) project superiorly and inferiorly from the pedicles, respectively.

With continued reference to FIG. 2, it can be seen that access to intervertebral spaces through access pathways 29 are limited from the posterior and transforaminal directions. The access is obstructed by portions of the vertebrae 4, such as the spinous processes 16, the articular processes 20, 22 and facets 24, 26. For example, the access pathways 29 in the transforaminal directions can be about 7 mm in width and 8 mm in height. Intervertebral implants that are small enough to fit through the limited access pathways 29 are usually too small to provide the necessary support for spinal restoration or fixation. Previous attempts to solve this problem have included cutting a portion of the vertebrae 4 to provide larger access pathways for larger intervertebral devices, such as removing the facets. However, in this application, new devices are disclosed that can fit through the access pathways 29 without cutting the vertebrae 4, and still provide sufficient support when implanted.

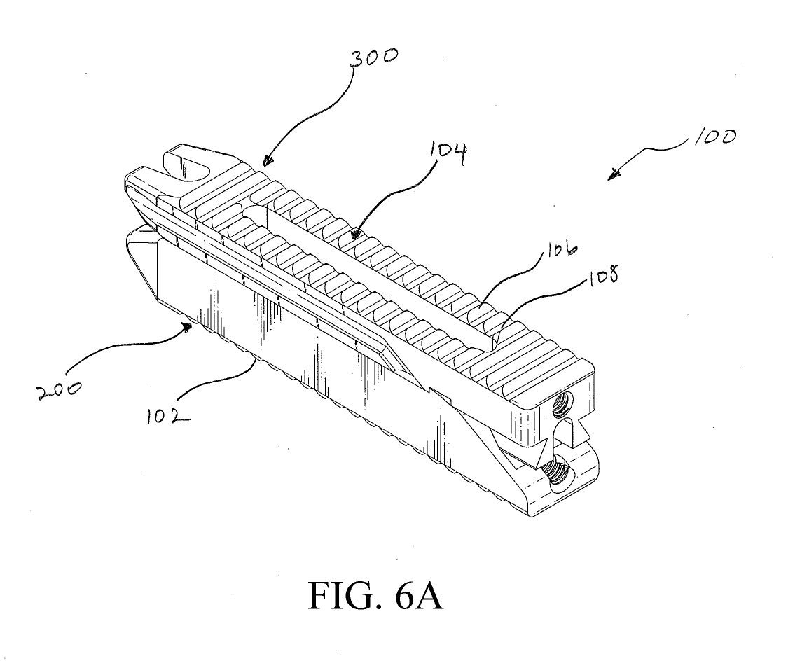

In this regard, FIGS. 6A-C illustrate an embodiment of an intervertebral implant 100 configured to be implanted using a minimally invasive procedure through restricted access pathways 29 in the vertebral column 2. The implant 100 can include a lower member 200, and an upper member 300 that is stacked on top of the lower member 200. In some embodiments, the implant 100 can include more than two members that are stacked on top of one another. For example, a third member can be stacked on top of the upper member 300 or below the lower member 200. In another, a third member can be positioned between the upper and lower members 300, 200.

As illustrated in FIGS. 6A-C, the implant 100 can have a bottom surface 102 and a top surface 104, which in some embodiments can be textured. In the illustrated embodiment, the surfaces 102, 104 include a plurality of ridges 106 and grooves 108 that extend perpendicular to the longitudinal direction of the implant 100. However, in other embodiments, the surfaces can have one or more of a variety of different features, such as for examples spikes or dimples. When the implant 100 is positioned in the intervertebral space, the bottom surface 102 can be disposed against or adjacent the inferior vertebra to help secure the bottom of the implant 100 with the vertebra. Conversely, the top surface 104 can be disposed against or adjacent the superior vertebra to secure the top of the implant 100. The textured features of the surfaces 102, 104 can advantageously promote osseointegration of the implant 100 with the vertebrae.

As will be described further below, the implant 100 can be inserted into an intervertebral space in a collapsed configuration and then changed to a stacked configuration. In the collapsed configuration, the lower member 200 can be separated from the upper member 300 to pass through the access pathway 29. As will be explained in detail below, in one embodiment the lower and upper members 200, 300 are inserted with one member in front of the other (e.g., sequentially) such that the insertion profile of the implant can approximate the height of an individual member of the implant 100.

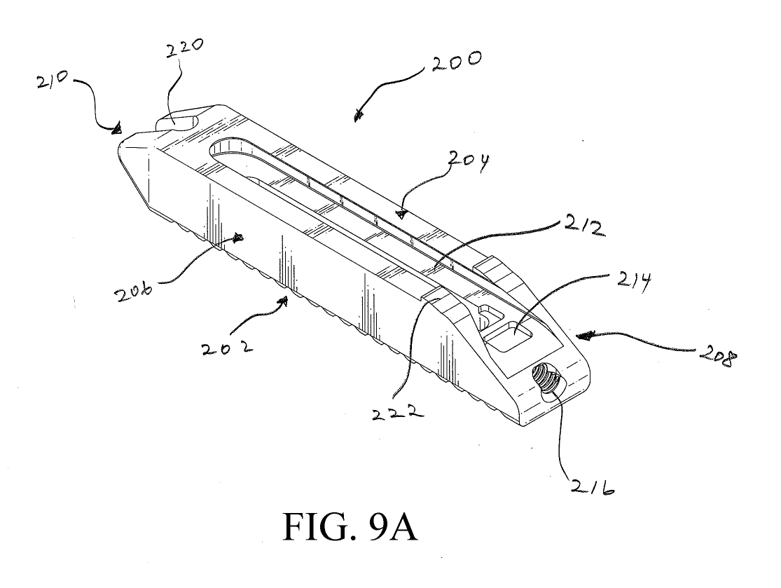

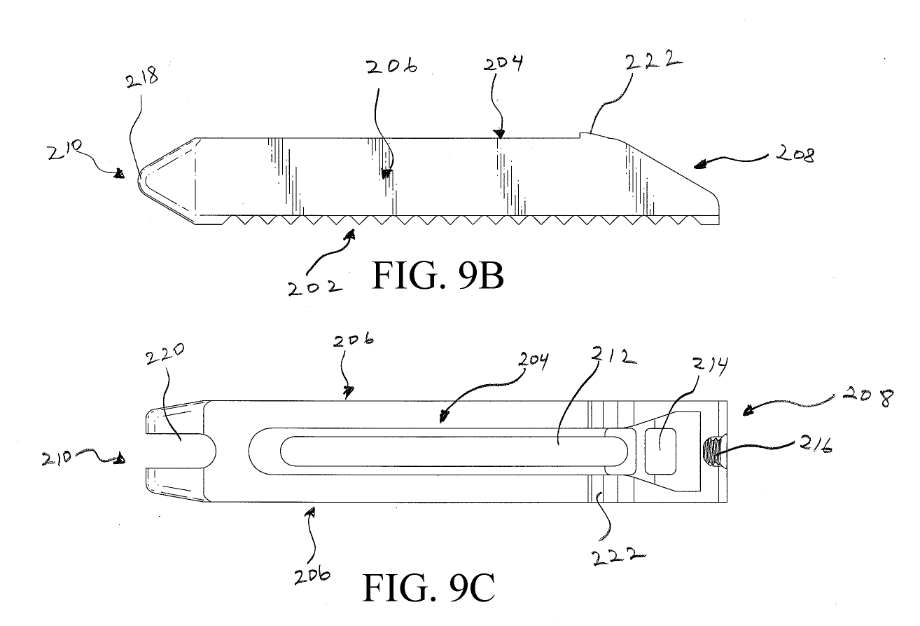

In the stacked configuration, the lower member 200 can be coupled to the top of the upper member 300. In some embodiments, the lower member 200 can have a first feature that is complementary to a second feature on the upper member 300, such that the first feature engages with the second feature to couple the lower member 200 with the upper member 300. For example, as illustrated in FIGS. 9A and 10A, the lower member 200 can have an elongate channel 212 that extends longitudinally along the top side 204 of the lower member 200. The upper member 300 can have a rail 312 that extends longitudinally along the bottom side 302 of the upper member 300 and which is complementary to the channel 212. The rail 312 can slideably engage with the channel 212 to couple the lower member 200 and the upper member 300. In other embodiments, the upper member can have more than one rail that slideably couple with complementary channels on the lower member.

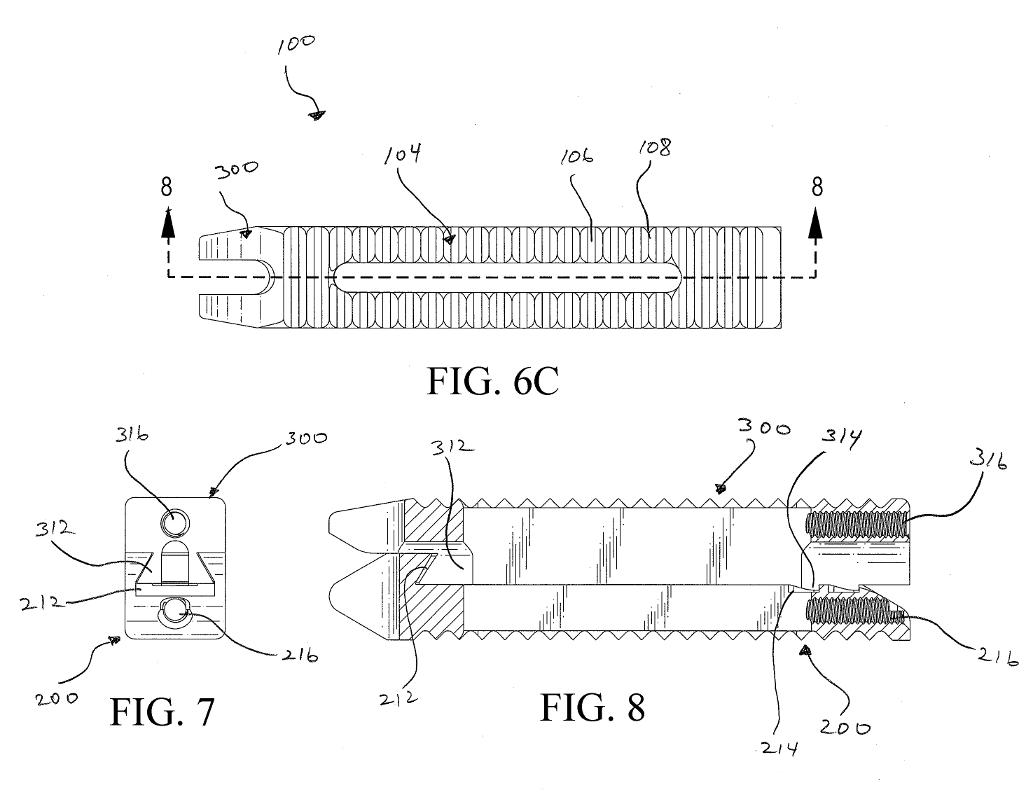

In some embodiments, the lower member 200 can have one or more depressions 214 that can accept one or more complementary protrusions 314 on the upper member 300. When the protrusions 314 are aligned with the depressions 214, as illustrated in FIG. 8, the lower member 200 and the upper member 300 can be locked together to prevent the upper member 300 from inadvertently disengaging from the lower member 200.

Accordingly, in the illustrated embodiment, the more complementary protrusions 314, elongate channel 212, and rail 312 of the upper and lower members 300, 200 can cooperate to limit or prevent lateral movement between the members 300, 200, vertical movement between the members 300, 200 and longitudinal movement between the members 300, 200. However, it should be appreciated that in modified embodiments, the members 200, 300 can be configured where one or more of these movements is permitted. In another embodiment, the lower and upper members 200, 300 can be formed without complementary structures that limit movement.

### Lower Member

The lower member 200 can be an elongate piece having a generally rectangular cross-section, as illustrated in FIGS. 9A-C. In other embodiments, the lower member can have a square cross-section, an oval cross-section, or any of a plurality of different types of cross-sectional shapes. In the illustrated embodiments, the lower member 200 has a bottom side 202, a top side 204 and two lateral sides 206. A rear side 208 is disposed on the proximal end of the lower member 200 and a front side 210 is disposed on the distal end.

In some embodiments, the width of the lower member 200 can be approximately 7 mm. In other embodiments, the width of the lower member 200 can be at least approximately 2 mm and/or less than or equal to approximately 12 mm. In still other embodiments, the width can be any other size beyond the identified preferred widths. The height of the lower member 200 can be approximately 6 mm, such that it can fit in the limited access pathways 29. In other embodiments, the height of the lower member 200 can be at least approximately 1 mm and/or less than or equal to approximately 7 mm. In still other embodiments, the height can be any other size beyond the identified preferred heights.

The bottom side 202 of the lower member 200 can be textured, as described above for the bottom surface 102. In the embodiment illustrated in FIGS. 9A-B, the bottom side 202 includes a plurality of ridges and grooves. In other embodiments, the textured surface can have one or more of a variety of different features, such as for examples spikes or dimples. The bottom side 202 is configured to abut against the native anatomy, such as the vertebrae, and secure the implant to the patient.

The top side 204 can be a generally flat surface having an opening of the channel 212, as explained below. The top side 204 can also include at least one wedge 222 that couples with a cutout on the upper member 300. The wedge 222 can have a tapered proximal side and a flat distal side. When the upper member 300 is slid onto the lower member 200, the tapered proximal side allows the upper member 300 to slide into the stacked position. When the final stacked position is reached, the flat distal side of the wedge 222 can help prevent the upper member 300 from uncoupling from the lower member 200. In other embodiments, the wedge can have any of a plurality of different shapes for acting as securement members.

In preferred embodiments, the rear side 208 is tapered. As best illustrated in FIG. 9B, the rear side 208 can increase in height in the proximal to distal direction. In some embodiments, the angle of the sloped rear side 208 is about 30°. In other embodiments, the angle of the rear side 208 can range from at least approximately 15° and/or less than or equal to approximately 60°. The sloped rear side 208 can provide a surface to guide the upper member 300 into proper position on top of the lower member 200 during coupling of the members, as explained further below.

The rear side 208 can include a bottom connector 216 for coupling a rod 224 or other elongate guide member. In the illustrated embodiment, the bottom connector 216 is a hole with internal threads for coupling to complementary outer threads on the rod 224. Conversely, in other embodiments, the bottom connector 216 can be a protrusion with external threads that couples to complementary internal threads on the rod 224. In some embodiments, the bottom connector 216 can have a shaped cavity for accepting a keyed rod such that the rod 224 can lock and unlock with the bottom connector 216 with a quarter or half turn. In another example, the bottom connector 216 can be a magnet or a ferrous material that attracts a magnet or ferrous material on the rod 224. In some embodiments, the bottom connector 216 can be any of a plurality of different types of connections that can couple to a complementary connector on the rod 224.

The front side 210 can have a tapered leading tip, as illustrated in FIGS. 9A-C. In the illustrated embodiment, the front side 210 has top and bottom sides that taper towards each other toward a rounded tip 218. The lateral sides of the front side 210 can also taper inward. The tapered front side 210 can advantageously help to insert through the restricted access pathway 29. The tapered shape can also advantageously deflect disc material or other material of the native anatomy as the lower member 200 is advanced into the intervertebral space. The rounded tip 218 can provide a blunt leading edge to help prevent injury to the native anatomy. In some embodiments, the front side 210 can have a front cavity 220, as illustrated in FIGS. 9A and 9C. The front cavity 220 advantageously provides increased surface area for improved integration of the lower member 200 to the native anatomy.

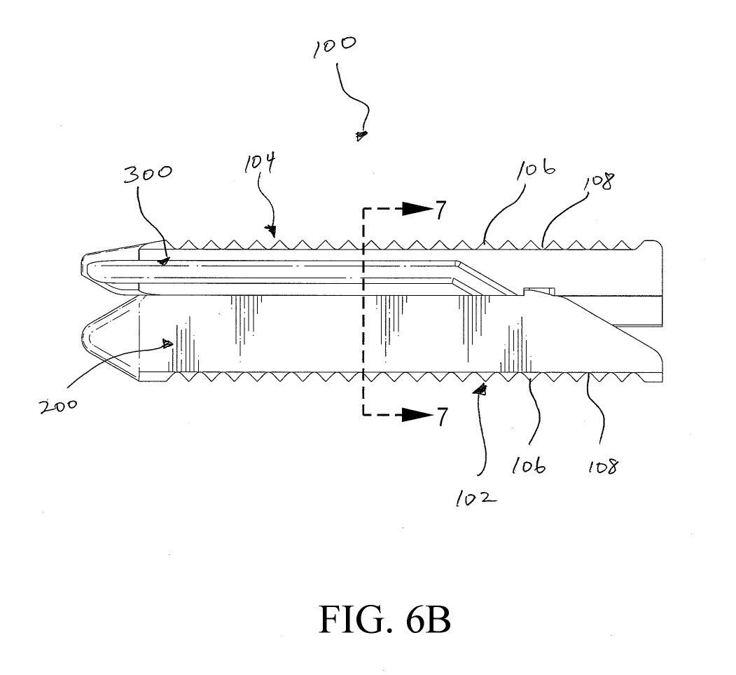

With continued reference to FIGS. 9A and 9C, a channel 212 can extend longitudinally through lower member 200 and is open to the top side 204 and rear side 208. The shape of the channel 212 can be configured for sliding engagement and locking engagement with the upper member 300. For example, as illustrated in FIG. 7, the cross-sectional shape of the channel 212 can be generally triangular. A complementarily shaped rail 312 of the upper member 300 can be slid into the channel 212 from the rear side 208. In some embodiments, the rear opening of the channel 212 can be tapered such that the opening is wider at the proximal end of the rear opening than the distal end of the rear opening. The tapered rear opening can help guide the upper member 300 into proper alignment with lower member 200. Once the channel 212 and rail 312 are coupled together, the lower member 200 and upper member 300 are prevented from vertical separation by the triangular shape of the channel 212. Although described as having a triangular cross-section, the channel can have other shapes that perform the same coupling results, such as circular or rectangular channel shapes.

In some embodiments, at least one depression 214 can be disposed in the channel 212. The depression 214 is configured to accept a protrusion on the upper member 300 for fixing the upper member 300 and the lower member 200 in the stacked configuration, as explained further below. In the illustrated embodiment, the lower member 200 has two depressions 214. Although illustrated as a generally rectangular depression, the shape can be any of a variety of shapes that can accept the protrusions on the upper member 300.

### Upper Member

With reference to an embodiment illustrated in FIGS. 10A-C, the upper member 300 can be an elongate piece having a cross-section with a generally rectangular top portion and a triangular bottom portion. In other embodiments, the upper member 300 can have cross-sectional portions that are generally square, oval, or any of a plurality of different shapes. In the illustrated embodiments, the upper member 300 has a bottom side 302, a top side 304 and two lateral sides 306. A rear side 308 is disposed on the proximal end of the upper member 300 and a front side 310 is disposed on the distal end.

In some embodiments, the width of the upper member 300 can be approximately 7 mm. In other embodiments, the width of the upper member 300 can be at least approximately 2 mm and/or less than or equal to approximately 12 mm. In still other embodiments, the width can be any other size beyond the identified preferred widths. The height of the upper member 300 can be approximately 6 mm, such that it can fit in the limited access pathways 29. In other embodiments, the height of the upper member 300 can be at least approximately 1 mm and/or less than or equal to approximately 7 mm. In still other embodiments, the height can be any other size beyond the identified preferred heights.

The top side 304 of the upper member 300 can be textured, as described above for the top surface 104. In the embodiment illustrated in FIGS. 10A-B, the top side 304 includes a plurality of ridges and grooves. In other embodiments, the textured surface can have one or more of a variety of different features, such as for examples spikes or dimples. The top side 304 is configured to abut against the native anatomy, such as the vertebrae, and secure the implant to the patient.

With continued reference to FIGS. 10A-C, the bottom side 302 can include a rail 312 that extends longitudinally along the upper member 300. The shape of the rail 312 can be configured for sliding engagement and locking engagement with the lower member 200. For example, as illustrated in FIG. 7, the cross-sectional shape of the rail 312 can be generally triangular. The rail 312 can be slid into a complementarily shaped channel 212 of the lower member 200 from the rear side 208. In some embodiments, the distal end of the rail 312 can be curved to help guide the upper member 300 into proper alignment with lower member 200. Once the channel 212 and rail 312 are coupled together, the lower member 200 and upper member 300 can be prevented from vertical separation by the triangular shape of the rail 312. Although described as having a triangular cross-section, the rail can have other shapes that perform the same coupling results, such as circular or rectangular rail shapes. In such embodiments, vertical separation of the lower member 200 and upper member 300 can be permitted.

In some embodiments, at least one protrusion 314 can be disposed in the bottom of the rail 312. The protrusion 314 is configured to fit in the depressions 214 on the lower member 200 for fixing the upper member 300 and the lower member 200 in the stacked configuration. In the embodiment illustrated in FIGS. 10A-C, the upper member 300 has four protrusions 314. The distal pair of protrusions 314 can fit in the distal depression 214 of the lower member 200 and the proximal pair of protrusions 314 can couple with the proximal depression 214 of the lower member 200. In some embodiments, the protrusion 314 can have a tapered distal side and a flat proximal side. When the upper member 300 is slid onto the lower member 200, the tapered distal side allows the rail 312 to slide into channel 212. When the final stacked position is reached, the protrusion 314 is positioned in the depression 214 and the flat proximal side of the protrusion 314 can help prevent the rail 312 from backing out from the channel 212. In other embodiments, the protrusion can have any of a plurality of different shapes for acting as securement members. In other embodiments, the protrusion 314 and the complimentary recess 214 on the lower member 200 can be eliminated. For example, the lower and upper member can be allowed to move longitududinally with respect to each other and/or be secured together with a different type of mechanism or a separate mechanism (e.g., a screw, stable, suture and/or adhesive).

In some embodiments, the bottom side 302 of the upper member 300 can have a bottom cavity 326. The bottom cavity 326 can advantageously provide increased surface area for improved integration of the upper member 300 with the lower member 200 and osseointegration with the native anatomy.

The bottom side 302 can also include at least one cutout 322 that couples with the wedge 222 on the lower member 200. The cutout 322 is illustrated as a generally rectangular depression on the bottom side 302; however, the cutout 322 can be of any of a variety of shapes and depths to complement the wedge 222 shape. When the final stacked position is reached, the wedge 222 on the lower member 200 can couple with the cutout 322 to help prevent the upper member 300 from uncoupling from the lower member 200. In some embodiments, the upper member 300 can have a wedge while the lower member 200 includes a corresponding cutout. In addition, as mentioned above, in certain embodiments, the cutout 322 and/or wedge 222 can be eliminated.

The rear side 308 of the upper member 300 can include a top connector 316 for coupling a rod 324 or other elongate guide member. In the illustrated embodiment, the top connector 316 is a hole with internal threads for coupling to complementary outer threads on the rod 324. Conversely, in other embodiments, the top connector 316 can be a protrusion with external threads that couples to complementary internal threads on the rod 324. In some embodiments, the top connector 316 can have a shaped cavity for accepting a keyed rod such that the rod 324 can lock and unlock with the top connector 316 with a quarter or half turn. In another example, the top connector 316 can be a magnet or a ferrous material that attracts a magnet or ferrous material on the rod 324. In some embodiments, the top connector 316 can be any of a plurality of different types of connections that can couple to a complementary connector on the rod 324.

In some embodiments, the front side 310 can have an angled front surface 318, as illustrated in FIG. 10B. The angled front surface 318 can be about 30°. In other embodiments, the angled front surface 318 can range from at least approximately 15° and/or less than or equal to approximately 60°. The angled front surface 318 can provide a sliding surface to guide the upper member 300 into proper position on top of the lower member 200 during coupling of the members, as explained further below.

In some embodiments, the front side 310 can be rounded. The rounded front side 310 can advantageously help to insert the upper member 300 through the restricted access pathway 29. The rounded shape can also advantageously deflect disc material or other material of the native anatomy as the upper member 300 is advanced into the intervertebral space. The rounded shape can provide a blunt leading edge to help prevent injury to the native anatomy. In some embodiments, the front side 310 can have a front cavity 320, as illustrated in FIG. 10C. The front cavity 320 advantageously provides increased surface area for improved integration of the upper member 300 to the native anatomy.

### Material

In some embodiments, the intervertebral body 100 can be made entirely of allograft bone (e.g., cortical bone). The use of allograft bone can beneficially promote integration of the intervertebral body 100 into surrounding tissue. However, as will be described in more detail below, other materials, or bioabsorbable or biocompatible materials can be utilized, depending upon the dimensions and desired in other embodiments. For example, in one embodiment, the intervertebral body 100 is substantially made entirely of allograft bone such that over 95% of the weight of the intervertebral body 100 is from allograft bone, in another embodiment, over 90% of the weight of the intervertebral body 100 is from allograft bone and in another embodiment over 75% of the weight of the intervertebral body 100 is from allograft bone. In such embodiments, the intervetabrabl body 100 can be formed of allograft bone and certain portions can be formed or coated with another biocompatible or bioabsorbable material, such as, a metal (e.g., titanium), ceramics, nylon, Teflon, polymers, etc.

In some embodiments, the intervertebral implant 100 can be fabricated autograph or other materials, or bioabsorbable or biocompatible materials can be utilized. Embodiments and components of the implant can be fabricated from metals such as titanium or synthetic materials are approved for medical use, such as Polyester Ester Ketone (PEEK) with hydroxyapatite. In some embodiments, the implant can comprise porous materials suitable to encourage osseointegration, such as for example allograft.

For example, in some embodiments, a resilient or elastic material, such as nylon or Teflon can be used. In such embodiments, a resilient lower member 200 and/or upper member 300 can allow the implant 100 to be compressible. The implant 100 can provide dynamic spacing, stabilization and support between adjacent vertebrae. The type of material used for the lower member 200 and/or upper member 300 can therefore be chosen depending on whether the implant 100 is intended to provide support at a given height or at a range of heights through compressibility of the implant 100. Moreover, the shape and size of the lower member 200 and/or upper member 300, as well as its material properties, can be dictated by the type of therapy desired. In addition, the material should be selected so as to ensure a minimum dimensional accuracy, resilience, and stability when the implant experiences loading in the stacked configuration.

### Method

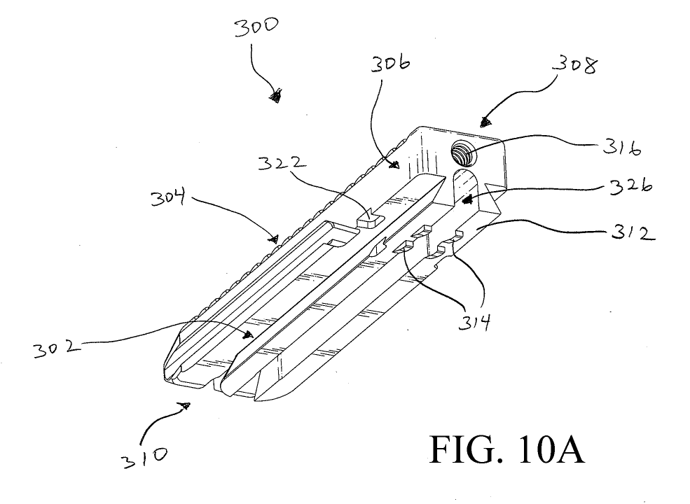

FIG. 11 illustrates the implant 100 in a collapsed configuration. The upper member 300 is positioned proximally and aligned longitudinally with the lower member 200. The implant 100 shown in FIG. 11 is in a minimal passing profile that allows the implant 100 to pass through limited access pathways 29 (see alos FIG. 2) and be placed at a desired intervertebral position for deployment. In some embodiments, the implant 100 can be manipulated through a minimally invasive access space created through a cannula. Thus, it is contemplated that embodiments disclosed herein can pass through a cannula or other type of access device to be implanted in the spine of a patient. In one embodiment, the implant 100 is inserted through a cannula that extends through the posterior natural access pathway 29 (see FIG. 2) available for accessing the disk space preferably without having to modify and/or enlarge this posterior natural access pathway. In other embodiments, the implant can be inserted from other directions and/or involve modifying or enlarging the pathway (e.g., with a drill or boring tool).

As discussed herein, the implant 100 can be maneuvered and operated using control tools, such as the rods 224, 324 illustrated in FIG. 11. As discussed above, the rear side 208 of the lower member 200 can have a bottom connector 216 that can be engaged by the rod 224. Similarly, the rear side 308 of the upper member 300 can have a top connector 316 that can be engaged by the rod 324. The lower member 200 and upper member 300 can be maneuvered into position in the intervertebral space and converted into the stacked configuration by manipulation of the rods 224, 324 through a minimally invasive incision, as discussed further below.

The implants disclosed herein can be implanted using a variety of surgical methods. In accordance with some embodiments, methods of implanting a stackable intervertebral implant are provided herein. Such methods can include one or more of the steps of dilating a pathway to an intervertebral disc, removing at least part of the nucleus of the intervertebral disc to define a disc cavity, scraping vertebral and plates from within the disc cavity, and deploying an intervertebral implant in the disc cavity.

In an implementation of the surgical methods disclosed herein, a surgeon can initiate dilation of a pathway to the intervertebral disc by using one of a variety of angles of approach. For example, a surgeon can use a posterior, posterolateral, or other angle of approach. In some embodiments, the surgeon can insert a needle to the intervertebral disc, such as a 18 G needle. The needle can define the pathway to the intervertebral disc. In this regard, the surgeon can then insert one or more dilators over the needle.

In some embodiments where dilators are employed, the surgeon can insert a first dilator over the needle and into or adjacent the intervertebral disc. The surgeon can then withdraw the needle completely while the first dilator remains in place. Next, the surgeon can insert a second dilator over the first dilator and into or adjacent the intervertebral disc. The second dilator can be configured to have a larger diameter than the first dilator. Subsequently, the surgeon can withdraw the first dilator completely while the second dilator remains in place. In some embodiments, additional dilators can be utilized to further dilate the pathway to the intervertebral disc. As such, the pathway can be dilated in a stepwise manner to minimize trauma. In some implementations, the first dilator can comprise an outer diameter of 3 mm and an inner diameter of 1 mm, and the second dilator can comprise an outer diameter of 6.3 mm and an inner diameter of 3.2 mm. Although the length of the dilators can vary, it is contemplated that the length of the dilators can be approximately 210 mm. Further, some implementations can utilize a guidewire having a diameter smaller than the inner diameter of the first dilator.

In accordance with some embodiments of the method, after the second dilator has been placed, the surgeon can insert a working sleeve over the second dilator. The working sleeve can be advanced over the second dilator until it is positioned adjacent to the intervertebral disc. It is contemplated that the working sleeve can be advanced such that a distal end of the working sleeve is positioned within the intervertebral disc. However, in some embodiments, the distal end can be positioned adjacent to or against the disc. In some embodiments, the working sleeve can have an inner diameter of 6.35 mm and an outer diameter of 9 mm. After the working sleeve is inserted, the second dilator can be removed.

The working sleeve is preferably configured to provide a sufficiently large interior geometry for advancing tools therein. For example, a trephine, crown reamer, and/or punch can be inserted through the working sleeve and used to remove the nucleus of the disc. In some embodiments, a second working sleeve can be advanced over the first working sleeve and positioned adjacent to or against the disc. The first working sleeve can then be removed. Accordingly, the second working sleeve can be configured with a larger inner and outer diameter than the first working sleeve. For example, the second working sleeve can have an inner diameter of 9.2 mm and outer diameter of 10 mm.

In accordance with some embodiments of the method, once the working sleeve is in place, an aperture or hole can be formed in the intervertebral disc by a drilling procedure. For example, a drill bit can be advanced into the disc in order to provide an intervertebral spacing approximately equal to the diameter of the drill bit. In this regard, the drill bit can have a diameter of approximately 9 mm. In some embodiments, the hole can be drilled into the end plates of the vertebrae as well as into the disc, thereby creating a space for the implant within the intervertebral space where the implant may have not otherwise been able to fit. In some cases, the creation of such a space in the intervertebral space may require not only drilling the disc, but also the end plates of the vertebrae. Further, it is also contemplated that other methods can be employed for removing the nucleus of the disc 6, such as for example using a punch and reamer.

In some embodiments, the method can further comprise using a rasp tool. The rasp tool can comprise an elongated body and one or more scraping components with an outer surface that is configured to scrape or create friction against the disc. For example, the outer surfaces can be generally arcuate and provide an abrasive force when in contact with the interior portion of the disc. In this manner, the rasp tool can prepare the surfaces of the interior of the disc by removing any additional gelatinous nucleus material, as well as smoothing out the general contours of the interior surfaces of the disc. The rasping may thereby prepare the vertebral endplates for fit with the implant as well as to promote bony fusion between the vertebrae and the implant. Due to the preparation of the interior surfaces of the disc, the placement and deployment of the implant may be more effective.

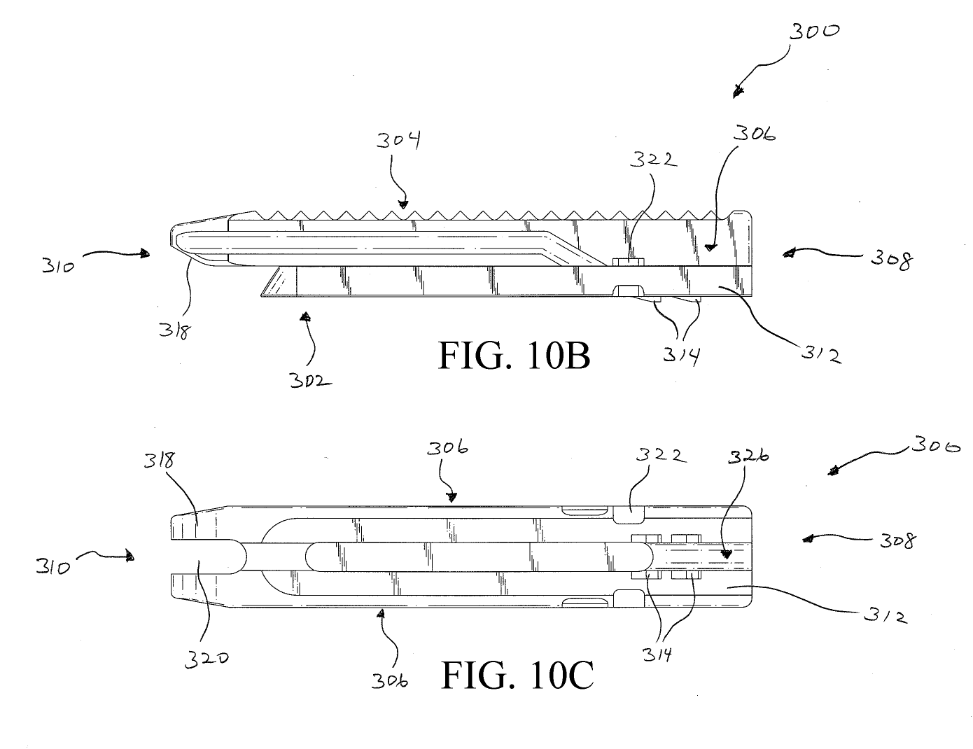

After the implant site has been prepared, the implant 100 can be advanced through the working sleeve 400 into the disc cavity, as illustrated in FIGS. 12-16. FIG. 12 illustrates a cross-sectional side view of adjacent vertebrae 4 with a working sleeve 400 adjacent an intervertebral space 7 and an implant 100 in a collapsed configuration inside the working sleeve 400. As illustrated, rods 224, 324 can be coupled to the lower member 200 and upper member 300, respectively, in order to position and to deploy the implant 100.

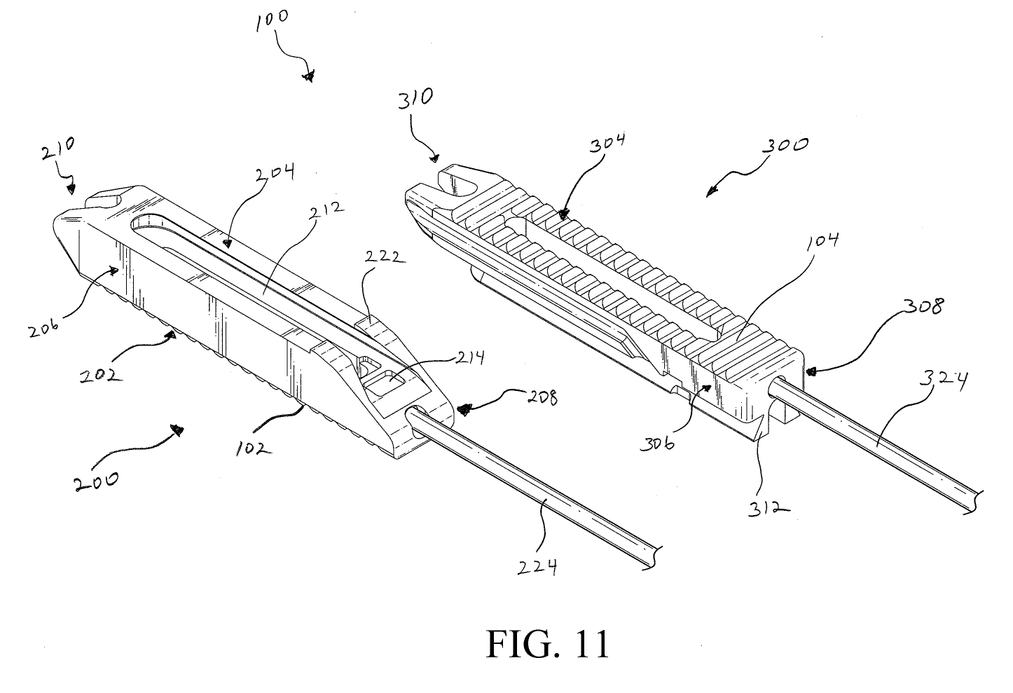

As illustrated in FIG. 13, the implant 100 can be inserted into the intervertebral space 7 by manipulation of the rods 224, 324. Preferably, the lower member 200 is inserted into the intervertebral space 7 first by pushing the rod 224 in the distal direction toward intervertebral space 7. The lower member 200 can be adjusted so that it is generally in its final implanted position. The upper member 300 can then be inserted into the intervertebral space 7 by distal movement of the rod 324, such that the upper member 300 engages with the lower member 200.

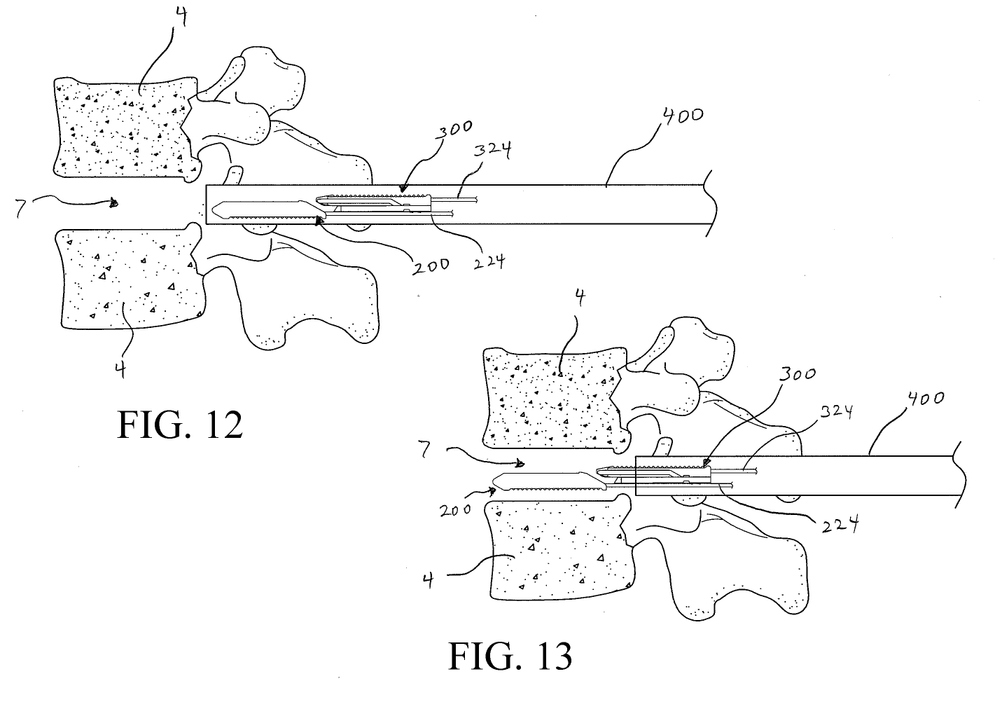

When the upper member 300 is moved toward the lower member 200, the two members can initially contact along complementary angled surfaces, as illustrated in FIG. 14. The rear side 208 of the lower member 200 can be inclined, as explained above, and the front side 310 of the upper member 300 can have an angled front surface 318. As the upper member 300 is thrust distally, the complementary angled surfaces can guide the upper member 300 upward, as illustrated in FIG. 14. Preferably, at least a portion of the rear side 208 of the lower member 200 and the angled front surface 318 of the upper member 300 are fabricated from a non-resilient or rigid material that facilitates slideable contact between the lower member 200 and the upper member 300.

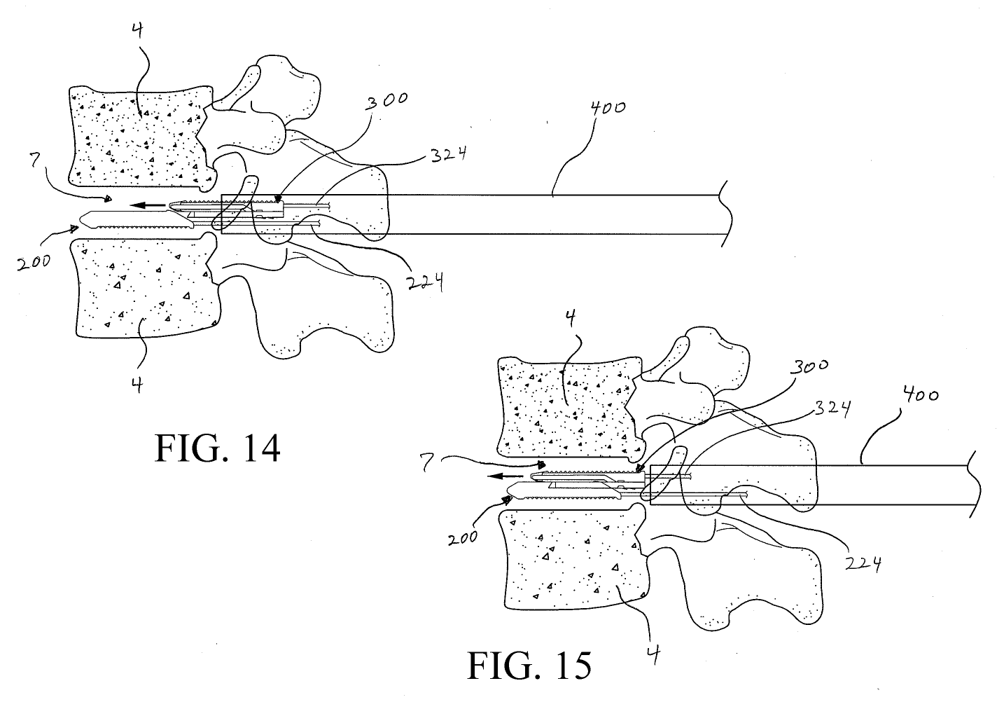

As the upper member 300 is thrust further in the distal direction, the rail 312 of the upper member 300 can be guided into proper engagement with the channel 212 of the lower member 200. As described above, the channel 212 can have a tapered rear opening and the rail 312 can have a curved distal end to help with alignment and engagement of the rail 312 in the channel 212. Continued movement of the upper member 300 in the distal direction can achieve increased slideable engagement of the rail 312 into the channel 212, as illustrated in FIG. 15. The lower member 200 and upper member 300 are preferably restricted from vertical (i.e., superior-inferior direction) movement relative to each other once the rail 312 is in slideable engagement with the channel 212. In some embodiments, the triangular or angled shape of the rail 312 and channel 212 can restrict the relative vertical movement, as described above.

In some embodiments, when the upper member 300 reaches a fully engaged position with the lower member 200, the protrusions 314 can fit within the depressions 214 to secure the implant 100 in a stacked configuration. Furthermore, in some embodiments, the wedges 222 can couple with the cutout 322 to provide further securement. In some embodiments, the implant 100 can be configured to be able to hold in one or more intermediate positions. For example, the upper member 300 can have protrusions 314 along various points along the longitudinal length of the rail 312 that can engage with the depressions 214 when the upper member 300 is in an intermediate position with the lower member 200.

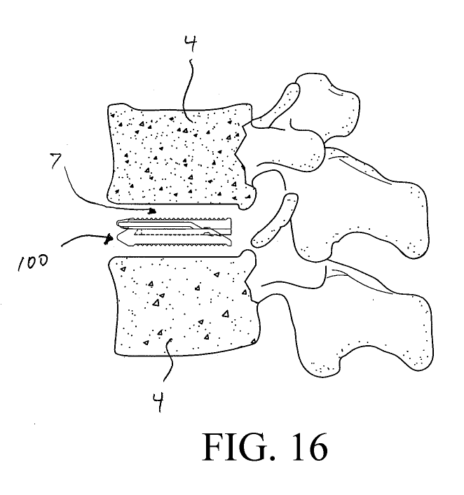

FIG. 16 illustrates the implant 100 in a final stacked configuration. In the illustrated embodiment, the implant 100 does not contact the inferior or superior vertebra. In other embodiment, the implant 100 can have any desired height and can contact one or both of the adjacent vertebrae.

After the implant 100 is positioned in the intervertebral space 7 in the desired position and orientation, the rods 224, 324 can be detached from the lower member 200 and upper member 300 and removed from the patient through the working sleeve 400. For example, in the case where the rods 224, 324 are attached with threaded engagement with the upper member 200 and lower member 300, the rods 224, 324 can be rotated to unfasten the threads.

In some embodiments, filler material can be inserted into the intervertebral space 7, for example in the hole formed during the drilling procedure. In some embodiments, the filler material can be inserted after the implant 100 is in its final position. In some embodiments, the filler material can be inserted into the intervertebral space 7 before the implant 100 is implanted. The filler material can help to fill in the gaps in the implant 100 and between the implant 100 and native anatomy. For example, the filler material can be inserted into the bottom cavity 326, which can help fuse the lower member 200 and the upper member 300. Furthermore, the filler material can advantageously help the implant 100 to provide additional dynamic support between vertebral bodies and promote osseointegration of the implant 100 with the vertebrae. Some examples of filler material include BMP, allograft and cement material.

After the implant procedure is complete, the working sleeve 400 can be withdrawn from the patient and the implant site can be closed.

In the figures, the elements have been represented in a schematic way in areas to facilitate conceptual understanding. For example, the tools that can be utilized to implant the device and otherwise perform the method have been particularly schematic, since these depend not only on the concrete realization of the implant, but the design and shape of the rest of the instruments being used. Obviously, there are numerous alternatives to what is shown.

Although these devices and methods have been disclosed in the context of certain preferred embodiments and examples, it will be understood by those skilled in the art that the present application extend beyond the specifically disclosed embodiments to other alternative embodiments and/or uses of the devices and obvious modifications and equivalents thereof. In addition, while several variations of the devices have been shown and described in detail, other modifications, which are within the scope of this application, will be readily apparent to those of skill in the art based upon this disclosure. It is also contemplated that various combination or sub-combinations of the specific features and aspects of the embodiments may be made and still fall within the scope of the present disclosure. It should be understood that various features and aspects of the disclosed embodiments can be combined with or substituted for one another in order to form varying modes of the disclosed devices. Thus, it is intended that the scope of at least some of the devices herein disclosed should not be limited by the particular disclosed embodiments described above.

What is claimed is:

1. An intervertebral implant comprising:
a first member comprising at least one channel extending along a longitudinal axis of the first member, the at least one channel being open to a top side and rear side of the first member, the first member further comprising a rear side having an angled surface; and
a second member comprising a bottom side having at least one rail extending along a longitudinal axis of the second member, the at least one rail configured for slideable engagement with the at least one channel, the second member further comprising a front side with an angled surface;
wherein the first and second members are formed substantially entirely of bone allograft.

2. The implant of claim 1, further comprising a rigid rod releasably coupled to the first member and/or second member.

3. The implant of claim 1, wherein the angled surface on the rear side of the first member is configured for sliding abutment with the angled surface of the front side of the second member.

4. The implant of claim 1, wherein the first member further comprises a bottom side with a textured surface.

5. The implant of claim 1, wherein the second member further comprises a top side with a textured surface.

6. The implant of claim 1, wherein the first member and/or second member has a generally rectangular cross-sectional shape.

7. The implant of claim 1, wherein the rail and channel have a generally triangular cross-sectional shape.

8. The implant of claim 1, the first member further comprising at least one depression and the second member further comprising at least one protrusion, wherein the at least one protrusion is configured to fit in the at least one depression in a secure engagement.

9. The implant of claim 1, further comprising at least one wedge disposed on the top side of the first member and further comprising at least one cutout on the bottom side of the second member, wherein the at least one wedge is configured to secure in the at least one cutout when the first member and second member are in the final stacked configuration.

10. The implant of claim 1, further comprising at least one additional member configured for stacking engagement with the first or second member.

11. The implant of claim 1, wherein the first member and/or second member is made at least partially of bone allograft.

12. The implant of claim 1, wherein a height of the first member measured as a distance from the top side to a bottom side of the first member is less than or equal to approximately 7 mm.

13. The implant of claim 1, wherein a height of the second member measured as a distance from a top side to a bottom of the rail of the second member is less than or equal to approximately 7 mm.

14. An intervertebral implant comprising:
a first member comprising at least one channel extending along a longitudinal axis of the first member; and
a second member comprising at least one rail extending along a longitudinal axis of the second member, the at least one rail configured for slideable engagement with the at least one channel.

15. The implant of claim 14, further comprising a rigid rod releasably coupled to the first member and/or second member.

16. The implant of claim 14, wherein the first member further comprises a rear side having an angled surface and the second member further comprises a front side with an angled surface, wherein the angled surface on the rear side of the first member is configured for sliding abutment with the angled surface of the front side of the second member.

17. The implant of claim 14, wherein the first member and/or second member has a generally rectangular cross-sectional shape.

18. The implant of claim 14, wherein the rail and channel have a generally triangular cross-sectional shape.

19. The implant of claim 14, further comprising at least one additional member configured for stacking engagement with the first or second member.

20. The implant of claim 14, wherein the first member and/or second member is made entirely of allograft.

21. The implant of claim 14, wherein a height of the first member measured as a distance from a top side to a bottom side of the first member is less than or equal to approximately 7 mm.

22. The implant of claim 14, wherein a height of the second member measured as a distance from a top side to a bottom of the rail of the second member is less than or equal to approximately 7 mm.

23. A method of implanting a stackable intervertebral implant, the method comprising the steps of:
inserting a first member made substantially of bone allograft of the implant into the disc cavity;
inserting a second member made substantially of bone allograft of the implant into the disc cavity so that it slideably engages with the first member of the implant in a stacked configuration.

24. The method of claim 23 further comprising dilating a pathway to an intervertebral space and removing at least a portion of an intervertebral disc to define a disc cavity

25. The method of claim 24, wherein the step of dilating comprises:
inserting a needle to the intervertebral space;
inserting a first dilator over the needle to the intervertebral space;
removing the needle;
inserting a second dilator over the first dilator to the intervertebral space;
removing the first dilator;
inserting a working sleeve over the second dilator to the intervertebral space; and
removing the second dilator.

26. The method of claim 23, further comprising the step of deploying at least one additional member of the implant so that it engages with the first or second member of the implant in a stacked configuration.

27. The method of claim 23, further comprising the step of removing rods that are releasably coupled to the first member and second member.

28. The method of claim 23, further comprising the step of inserting filler material into the disc cavity before or after the steps of deploying the first and second members.

29. The method of claim 28, wherein the filler material is allograft.
